(19) United States
(12) Patent Application Publication
(10) Pub. No.: US 2026/0106035 A1
(43) Pub. Date: Apr. 16, 2026

(54) Machine Learning System for Adult Oncology Patients Receiving Multimodal Cancer Therapy

(71) Applicant: Yun Jeong Seo, Queens, NY (US)

(72) Inventor: Yun Jeong Seo, Queens, NY (US)

(21) Appl. No.: 18/912,480

(22) Filed: Oct. 10, 2024

(51) Int. Cl. G16H 50/30; G16H 10/60; G16H 40/20; G16H 50/20

(52) U.S. Cl. CPC G16H 50/30; G16H 10/60; G16H 40/20; G16H 50/20

(57) ABSTRACT

The HEART (High ED/Admission Risk Therapy) Team Protocol is an innovative, evidence-based solution designed to reduce the number of preventable emergency department visits and unplanned hospital admissions among adult oncology patients undergoing multimodal cancer therapy. The protocol's structured nursing assessment tool (the HEARTS checklist) and decision-support algorithm (the HEART algorithm) enable early detection of complications, allowing healthcare providers to take timely, appropriate action based on real-time clinical data. The multidisciplinary approach ensures that patients receive comprehensive care, addressing both medical and psychosocial needs.

### BACKGROUND OF THE INVENTION

### Field of Invention

The present invention relates to the field of healthcare, specifically within oncology departments, focusing on high-risk adult oncology patients undergoing multimodal cancer therapies such as chemotherapy, radiation therapy, and other combined treatment modalities. The invention centers on a system and method for managing these patients effectively by reducing unnecessary emergency department (ED) visits and unplanned hospital admissions. This is achieved through the implementation of a protocol, which includes a structured clinical assessment tool, and a decision-support algorithm designed to guide healthcare providers in making real-time interventions based on a patient's risk level.

The invention specifically addresses challenges faced by healthcare providers in detecting early signs of complications in cancer patients and aims to improve patient outcomes, reduce healthcare costs, and ensure efficient resource utilization in hospital settings. The protocol integrates interdisciplinary collaboration among nurses, oncologists, social workers, and other members of the healthcare team, enabling holistic care for patients at risk of complications arising from multimodal cancer therapy.

### Brief Summary of The Invention

The HEART (High ED/Admission Risk Therapy) Team Protocol is a computer-implemented evidence-based system developed to reduce the number of unnecessary emergency department visits and unplanned hospital admissions among adult oncology patients undergoing multimodal cancer therapy, such as concurrent chemoradiation therapy. The protocol employs two primary tools: a structured nursing assessment checklist (the HEARTS checklist) connected to an electronic health record and an algorithmic decision-support tool (the HEART algorithm), both of which are designed to enable early detection of complications that could otherwise lead to emergency medical interventions.

The HEARTS checklist provides nurses with a standardized approach to patient assessment, focusing on critical symptoms and physiological changes that may indicate early complications, such as dehydration, fatigue, oral intake challenges, and altered vital signs. The HEART algorithm supports real-time decision-making by categorizing patients into different risk levels (mild, moderate, or severe) based on the data collected from the checklist. This enables nurses to take immediate action, including managing symptoms, coordinating referrals to a multidisciplinary care team, or escalating cases to emergency services.

Through multidisciplinary collaboration, the HEART protocol ensures that patients receive comprehensive, timely, and individualized care. The system is designed to be easily integrated with electronic health record (EHR) systems, allowing for seamless documentation and coordination across healthcare teams. This protocol not only reduces the likelihood of preventable ED visits and hospital admissions but also improves patient outcomes and streamlines care delivery for oncology patients receiving multimodal therapy.

### BRIEF DESCRIPTION OF THE DRAWINGS

FIG. 1 illustrates a process according to an embodiment of the present invention.

FIG. 2 illustrates a HEARTS assessment checklist.

FIG. 3 illustrates a HEARTS team protocol algorithm.

### DETAILED DESCRIPTION

Oncology patients undergoing multimodal cancer therapies are at high risk of developing complications that may lead to emergency medical interventions. These complications include, but are not limited to, dehydration, electrolyte imbalances, infection, severe fatigue, nausea, and vomiting—conditions that are often exacerbated by the aggressive nature of combined chemoradiation therapy. In many cases, these complications are not detected early, leading to preventable ED visits and unplanned hospital admissions, which disrupt cancer treatment schedules, increase healthcare costs, and negatively impact patient quality of life.

The current approaches to managing high-risk cancer patients often rely on subjective assessments, which are inconsistent and prone to human error. Moreover, many healthcare providers lack the tools and structured protocols necessary to identify early warning signs of complications, leading to delays in intervention. This delay not only burdens healthcare systems with overcrowded EDs but also places additional stress on patients and their families.

The HEART Team Protocol was developed to address these shortcomings by providing healthcare providers—particularly nurses—with a standardized, evidence-based framework for assessing and managing high-risk oncology patients. By introducing a structured assessment tool (the HEARTS checklist) and an algorithmic decision-making process (the HEART algorithm), the protocol enables early detection and timely intervention, reducing the likelihood of complications escalating to the point of requiring emergency care.

The HEARTS checklist is the primary assessment tool used by nurses to evaluate the risk level of oncology patients undergoing multimodal therapy. The checklist is designed to capture critical clinical data, including changes in vital signs, patient-reported symptoms, and physical exam findings that may indicate early signs of complications. Key components of the checklist include:

Vital Signs Monitoring: Continuous monitoring of vital signs, such as blood pressure, heart rate, respiratory rate, and temperature, to detect deviations from baseline values that may signal dehydration, infection, or cardiovascular stress.

Oral Intake Assessment: Evaluation of the patient's ability to maintain adequate hydration and nutrition, focusing on symptoms such as difficulty swallowing, reduced appetite, or nausea, which can contribute to dehydration and electrolyte imbalances. These can be assessed through body weight monitoring, lab values, and vital signs such as orthostatic measurements.

Symptom Tracking: Regular documentation of symptoms commonly associated with cancer treatment side effects, including fatigue, nausea, vomiting, dizziness, and pain. These symptoms are critical indicators of the patient's overall condition and risk level. Monitoring begins on day 1 of chemoradiation treatment, followed by at least bi-weekly HEART assessments, continuing through the last day of treatment. This helps track the patient's condition trends and allows for immediate actions based on the outcomes of the HEART assessments, following the HEART team algorithm.

Activity Level and Fatigue Assessment: Assessment of the patient's functional status, including their ability to perform activities of daily living (ADLs). A significant decline in activity level may indicate worsening fatigue or physical deterioration, necessitating intervention, such as referring to social workers to provide eligible support.

Psychosocial and Support Needs: Evaluation of the patient's mental health, social support systems, and the need for additional resources, such as home health aides, nutrition counseling, or transportation service.

By using the HEARTS checklist, nurses are able to perform comprehensive, standardized assessments that ensure consistency in patient evaluations. This not only improves the accuracy of risk detection but also facilitates more efficient communication between the nursing staff and other members of the care team.

The HEART algorithm is a decision-support tool that processes the data collected through the HEARTS checklist and categorizes patients into different risk levels—mild, moderate, or severe—based on predefined criteria. The algorithm uses a combination of clinical data points, such as vital sign trends, symptom severity, and overall functional status, to generate recommendations for care. For example:

Mild Risk: Patients categorized as mild risk may be managed with outpatient interventions, such as increased hydration, nutritional support, or adjustments to their symptom management plan. The nurse may continue to monitor the patient closely and schedule a follow-up assessment within 24-48 hours to reassess the patient's condition.

Moderate Risk: Patients at moderate risk may require more immediate interventions, such as intravenous (IV) hydration or referrals to specialists (e.g., physicians, nurse practitioners, and a nutritionist or social worker) to address specific issues related to oral intake, fatigue, or psychosocial needs. The algorithm may recommend that the nurse consult with the oncology team or the multidisciplinary care team to determine the best course of action.

Severe Risk: Patients classified as severe risk may need critical care, including potential referral to the emergency department for treatment of conditions such as dehydration, infection, or severe electrolyte imbalances. The algorithm triggers an alert, prompting the nurse to escalate care to the attending physician or initiate emergency medical services (EMS) if necessary.

The HEART algorithm is designed to function in real-time, providing immediate feedback to the nurse based on the patient's clinical data. This enables nurses to act quickly and confidently, ensuring that patients receive the appropriate level of care based on their individual risk factors.

One of the key innovations of the HEART Team Protocol is its emphasis on multidisciplinary collaboration. Cancer patients often have complex needs that extend beyond the scope of oncology treatment, including nutritional support, mental health services, and social support. The HEART protocol ensures that patients receive holistic care by promoting seamless communication and collaboration between healthcare providers from different specialties.

Oncologists/Radiation Oncologists/Nurse Practitioner: Provide medical oversight and adjust treatment plans based on the patient's condition and response to therapy.

Nurses: Serve as the primary point of contact for patient assessments and interventions, utilizing the HEARTS checklist and algorithm to guide care. Social Workers: Address psychosocial issues, such as access to care, transportation, and emotional support. Social workers also help patients navigate financial challenges related to their treatment.

Nutritionists: Provide dietary counseling to help patients maintain adequate nutrition and hydration during treatment, which is critical for preventing complications like dehydration and malnutrition.

Pharmacists: Offer medication management services, ensuring that patients adhere to prescribed therapies and receive appropriate support for managing side effects.

By integrating these professionals into a cohesive team, the HEART protocol fosters comprehensive care planning that addresses all aspects of the patient's health. Regular team meetings and EHR integration ensure that all members of the team are kept up to date on the patient's status, allowing for coordinated and efficient care.

The HEART Team Protocol is designed to be fully integrated with electronic health records (EHR) systems, enabling seamless documentation of patient assessments, interventions, and outcomes. EHR integration allows for real-time data sharing across the multidisciplinary care team, ensuring that all providers have access to up-to-date information about the patient's condition.

Improved Care Coordination: By having all patient data stored in a central location, providers can easily communicate and collaborate on patient care. This reduces the likelihood of miscommunication or delays in care.

Efficient Documentation: The HEARTS checklist and algorithm-generated recommendations are automatically documented in the EHR, reducing the administrative burden on nurses and ensuring that all assessments are accurately recorded.

Data Analytics: The protocol's integration with EHR systems enables the collection of valuable patient data that can be analyzed to track trends, identify patterns, and evaluate outcomes over time. Healthcare providers can leverage this data to make evidence-based decisions, optimize patient management strategies, and continuously improve the effectiveness of the protocol. Additionally, healthcare administrators can use the analytics to measure the impact of the HEART protocol on hospital metrics, such as reduced ED visits, shortened hospital stays, and overall healthcare cost savings.

Compliance with Regulatory Guidelines: Integration with EHR ensures that all data collected is in compliance with relevant healthcare regulations, including those established by the Centers for Medicare & Medicaid Services (CMS) and other governing bodies. This compliance is critical in maintaining accreditation, ensuring patient safety, and adhering to best practices in oncology care.

The immediate benefits of the HEART Team Protocol are numerous and have a profound impact on both patient care and healthcare system efficiency. These benefits include:

Reduction in Unnecessary ED Visits: By providing evidence-based early detection of complications and enabling timely interventions, the HEART protocol helps reduce the number of unnecessary ED visits among oncology patients. This not only alleviates the burden on emergency departments but also minimizes disruptions to the patient's cancer treatment schedule.

Improved Patient Outcomes: Early detection and intervention prevent complications from escalating to severe levels, leading to better patient outcomes. Patients are less likely to experience treatment interruptions or hospital stays, which contributes to faster recovery and improved quality of life.

Increased Nurse Confidence: The structured nature of the HEARTS checklist and the decision-support provided by the HEART algorithm enhance nurses'ability to assess and manage high-risk cancer patients. Nurses are empowered to take immediate, evidence-based action, which increases their confidence in managing complex cases.

Enhanced Communication: The protocol fosters better communication between nurses, physicians, and other members of the healthcare team. The use of standardized tools ensures that all providers are on the same page, leading to more coordinated and efficient care delivery.

In the long term, the HEART Team Protocol generates sustainable improvements in oncology care, healthcare resource utilization, and patient satisfaction. Key long-term benefits include:

Cost Savings for Healthcare Systems: By reducing the frequency of unnecessary hospital admissions and ED visits, the protocol generates significant cost savings for healthcare providers. These savings can be reinvested into other areas of care, further enhancing the quality of services offered to patients.

Adherence to CMS Guidelines: The protocol helps oncology departments comply with CMS guidelines for evidence-based care, which is critical for maintaining funding and accreditation. Compliance with these guidelines also ensures that patients receive care that is aligned with the latest best practices in oncology.

Improved Continuity of Care: The HEART protocol reduces the likelihood of treatment disruptions, allowing patients to continue their cancer therapies with minimal interruptions. This continuity of care is essential for optimizing treatment outcomes, particularly in multimodal therapies that require strict adherence to treatment schedules.

Data-Driven Improvements: Over time, the data collected through the protocol can be used to refine and enhance the system. Healthcare providers can identify trends, evaluate the effectiveness of specific interventions, and adjust the protocol as needed to further improve patient outcomes and system efficiency.

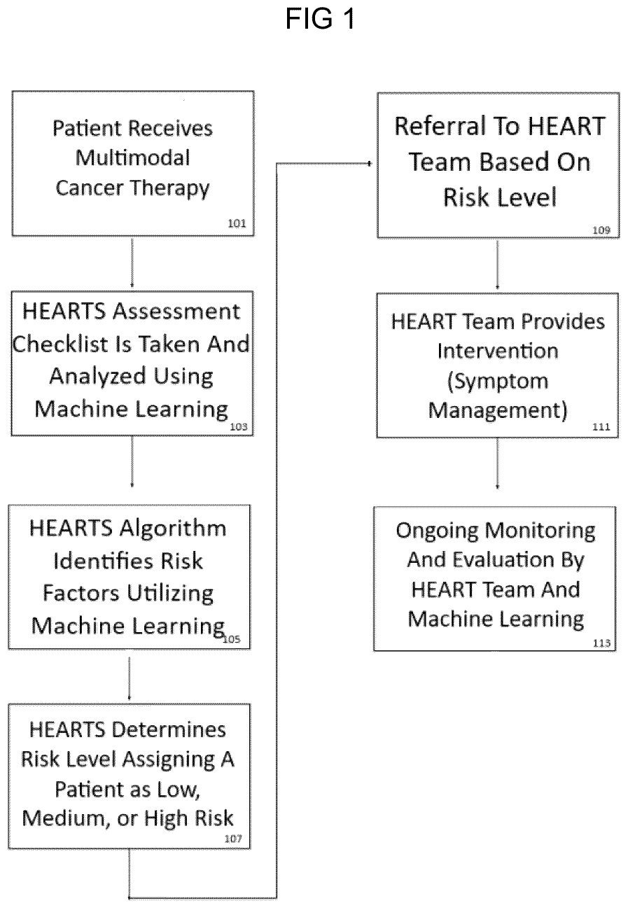

FIG. 1-101 : The patient undergoes a combination of treatments such as chemotherapy and radiation, which can increase the risk of complications. This phase marks the starting point for potential emergency department visits or unplanned hospital admissions.

FIG. 1-103: The nurse performs a structured assessment using the HEARTS checklist to evaluate the patient's symptoms, vital signs, and overall health status. This checklist helps identify early warning signs of complications, such as dehydration or severe fatigue. Results are inputted into a machine learning HEART algorithm. Machine learning algorithms are used to make a prediction or classification of a patient based on some input training data gleaned from past patients, which can be labeled or unlabeled. The algorithm will produce an estimate about a pattern in the data. An error function evaluates the prediction of the model. If there are known examples, an error function can make a comparison to assess the accuracy of the model. A model optimization process then occurs. If the model can fit better to the data points in the training set, then weights are adjusted to reduce the discrepancy between the known example and the model estimate. The algorithm will repeat this “evaluate and optimize” process, updating weights autonomously until a threshold of accuracy has been met. Supervised learning in particular uses a training set to teach models to yield the desired output. This training dataset includes inputs and correct outputs, which enables the model to learn over time. The algorithm measures its accuracy through the loss function, adjusting until the error has been sufficiently minimized.

FIG. 1-105: HEART algorithm is used to analyze the assessment results, identifying risk factors that may lead to an emergency department visit or hospital admission. The algorithm standardizes decision-making, ensuring prompt identification of patients at risk.

FIG. 1-107: The HEART algorithm categorizes the patient's risk level into low, moderate, or high. This classification informs the next steps in the care plan, determining the urgency and type of intervention required.

FIG. 1-109: If the risk is moderate or high, he patient is referred to the multidisciplinary HEART team, which includes oncologists, social workers, and other healthcare professionals. The team collaborates to provide targeted interventions, such as hydration, social support, or symptom management.

FIG. 1-111: The HEART team implements interventions based on the patient's specific needs as determine by the HEART algorithm and machine learning, addressing complications such as dehydration, nausea, or poor oral intake. Timely intervention reduces the likelihood of emergency department visits and unplanned hospitalizations.

FIG. 1-113: The HEART team continuously monitors the patient's condition, adjusting care as needed to prevent further complications. Regular evaluations ensure that the patient receives appropriate support throughout their cancer treatment, improving overall outcomes and reducing unnecessary admissions.

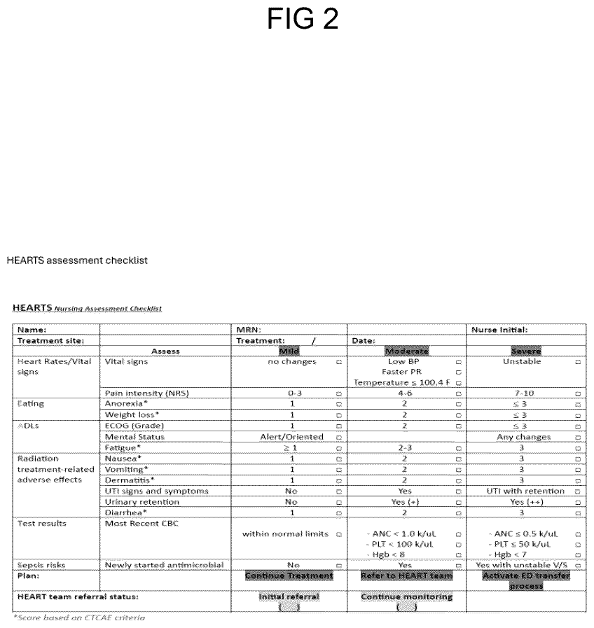

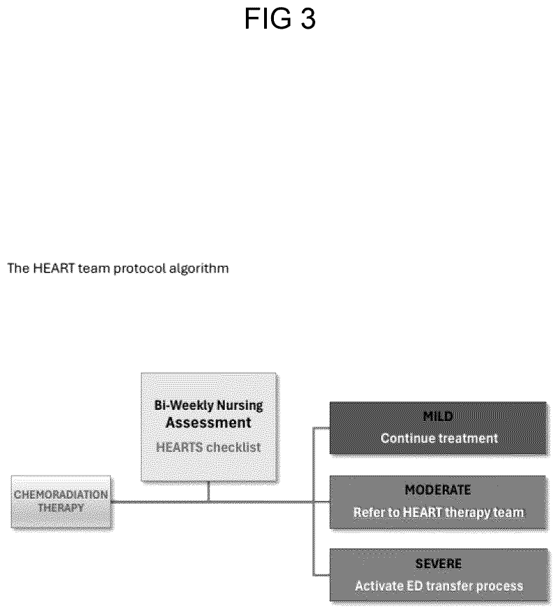

FIG. 2 and FIG. 3 illustrate the HEART assessment checklist and the protocol algorithm for use in the process outlined in FIG. 1. The protocol's structured nursing assessment tool (the HEARTS checklist) and decision-support algorithm (the HEART algorithm) enable early detection of complications, allowing healthcare providers to take timely, appropriate action based on real-time clinical data. The multidisciplinary approach ensures that patients receive comprehensive care, addressing both medical and psychosocial needs.

What is claimed is:

1. A system for managing high-risk oncology patients, comprising:
a. A structured nursing assessment tool (the HEARTS checklist) that captures vital clinical data, including changes in vital signs, oral intake, and symptom severity based on the CTCAE criteria, to detect early signs of complications in adult oncology patients receiving multimodal cancer therapy.
b. An algorithm-driven decision support tool (the HEART algorithm) that categorizes patients into mild, moderate, or severe risk levels based on clinical data and provides real-time recommendations for interventions, wherein the algorithm-driven decision support tool is implemented using a machine learning model on a computer server; and
c. One or more Integrations with electronic health record (EHR) systems to document patient assessments, interventions, and outcomes in real-time.

2. A method for reducing emergency department visits and hospital admissions in high-risk adult oncology patients, comprising:
a. Collecting patient data through a structured nursing assessment (the HEARTS checklist) that evaluates vital signs, symptoms, and oral intake.
b. Applying a decision-making algorithm (the HEART algorithm) that uses the collected data to determine the patient's risk level and recommend appropriate interventions.
c. Managing mild-risk patients through outpatient interventions, such as increased hydration, nutritional support, or symptom management.
d. Referring moderate-risk patients to a multidisciplinary care team for additional support, including social services, nutritional counseling, or specialized oncology care by physicians and nurse practitioners.
e. Escalating care for severe-risk patients by initiating emergency interventions, such as IV hydration, infection management, or transfer to the emergency department.

3. A system for improving care coordination and outcomes in oncology patients undergoing multimodal cancer therapy, comprising:
a. A decision-support algorithm that processes real-time clinical data and categorizes patients into risk levels, providing actionable recommendations for care.
b. A nurse-driven protocol that empowers nursing staff to initiate early interventions and refer patients to a multidisciplinary care team based on clinical findings.
c. A multidisciplinary care model that integrates the expertise of oncologists, social workers, nutritionists, and other healthcare providers to deliver comprehensive, patient-centered care.
d. A system for integrating patient data into EHRs, allowing for seamless documentation of assessments, interventions, and outcomes, and enabling data analytics for continuous improvement of care.
